(19) United States
(12) Patent Application Publication
(10) Pub. No.: US 2003/0236540 A1
(43) Pub. Date: Dec. 25, 2003

(54) Ear cleaning device

(76) Inventors: Jorge Mendez, Lima (PE); Paul A. Mullin, Littleton, CO (US)

Correspondence Address: The Law Office of Craig W. Barber, P.O. Box 16220, Golden, CO, 80402-6004, US

(21) Appl. No.: 10/179,143

(22) Filed: Jun. 24, 2002

(51) Int. Cl. A61F 9/00

(52) U.S. Cl. 606/162

(57) ABSTRACT

The present invention teaches an improved ear cleaner consisting of one piece of solid flat plastic, inexpensively molded or stamped to an elongate oval shape with one aperture at each end to form a loop. The present invention teaches that an improved ear cleaner may be made of a length such that it becomes difficult for the user to damage their ear drum. The invention further teaches that an improved ear cleaner may be made having a rounded shape and a soft consistency such that should the ear cleaning head (a thin and soft loop) impact the ear drum, it will bend rather than injure the ear drum. The present invention further teaches a method of manufacturing the ear cleaner which provides a very inexpensive method of making a one piece ear cleaner requiring no further assembly after manufacture. The present invention further teaches that the ear cleaner may advantageously be stamped rather than molded for further decrease in cost of manufacture.

### FIELD OF THE INVENTION

This invention relates to generally to ear cleaning devices and more specifically to planar (flat) disposable plastic ear cleaning devices.

### BACKGROUND OF THE INVENTION

Ear cleaning devices for both personal and clinical use in general require the insertion of some sort of cleaning head into the ear of the user/patient. This is more hazardous than is generally understood, as the cleaning head can come into contact with the ear drum, resulting in substantial damage. As a result, under modern practice US doctors actually discourage patients from cleaning their own ears.

Cost is another issue, as such ear cleaning devices should be either reusable (which requires cleaning between uses, an activity which substantially increases the cost of using the device) or else must be inexpensive and disposable.

U.S. Pat. No. 6,270,510 issued Aug. 7, 2001 to Westendorf, for “NOSTRIL GROOMING TOOL” discloses a disposable device having a semi-flexible shaft-like body and padded eyelets. It appears to be made out of twisted paper bound to provide strength, but also appears to be long enough to impact an ear drum.

US Patent Application Publication No. US2001/0001828 A1 published May 24, 2001 for “EAR HYGIENE DEVICE”, inventor Begun; discloses a double-headed ear cleaner having an open, toothed loop at one end and an absorbent material in a loop at the other end. The overall construction is complex and the handle area of a three dimensional shape requiring injection molding.

U.S. Pat. No. 5,888,199 issued Mar. 30, 1999 to Karell et al for “EAR CLEANING DEVICE WITH A FLEXION PART” discloses a loop mounted on a flexible portion of the device. The overall construction is complex and three dimensional, requiring both injection molding and assembly.

U.S. Pat. No. 5,715,850 issued Feb. 10, 1998 to Markgraaf for “PERSONAL EAR CLEANING DEVICE” teaches another device having a looped end and a complex three dimensional body capable of accepting different cleaning heads.

U.S. Pat. No. 5,632,756 issued May 27, 1997 to Kruglick for “EAR CLEANING DEVICE UTILIZING BULBOUS BANDED CAGE” discloses a cylindrical shaft for the body and a three-dimensional loop or cage for the cleaning implement, with a cotton swab at the other end. The device is supposed to be disposable.

U.S. Pat. Nos. 5,509,921 (Apr. 23, 1996) and 5,334,212 (Aug. 2, 1994) issued to Karell for “SAFE EAR WAX REMOVER” and “EAR WAX EXTRACTOR WITH DEPTH CONTROL” disclose awareness of the problem of depth control, but both disclose the handle of the devices connected to a very long shaft. In both cases, the shaft is necessary for operation of a safety stopper which slides up and down the handle. Obviously, these devices require assembly as part of the manufacture process, and may be misused by the end user so as to damage their eardrum.

U.S. Pat. No. 5,209,757 issued May 11, 1993 to Krug et al for “ILLUMINATED EAR CLEANING DEVICE” teaches an ear cleaning device with a round handle able to hold a battery and wiring, and a small light bulb within the cleaning loop. This patent also discloses a long shaft connecting handle and loop. The device is obviously not disposable, requiring wiring, light bulb, battery, handle, magnifier, body, lense support and loop to be assembled.

U.S. Pat. No. 3,203,418 issued Jun. 20, 1962 to Johnston is an interesting patent entitled “EAR SWABS”. The '418 patent teaches the use of a metal safety-pin-like-body to hold a padding tubular cleaning swab. This patent discloses an elongated wire body having a partial loop at one end, and appears to have a disposable component. The tubular disposable component is slipped over a safety pin or the special holder. The wire body is metal and apparently long enough to pose a potential danger to the ear drum, while its construction appears to be bent wire. Stamping such a body appears impossible, even before the tubular swab is slipped onto the wire body.

Five older patents disclose eye cleaning devices of complex construction having loops and circular bodies: U.S. Pat. Nos. 1,396,309; 1,263,942; 950,065; 772,177; and 462,910. Issued to Bingman, ('309); Schroeder ('942); Alexander ('065); Ramus ('177) and Hallock ('910), all show metal devices having loops which are apparently to be inserted in the eye or ear of the user to remove debris therefrom. Hallock '910 shows an elongate flat “jack-knife” construction. None disclose bodies which might be stamped out of plastic at low cost nor structural similarities to the present invention, in addition to concerning the eye and not the ear.

U.S. Pat. No. 147,660 issued Feb. 17, 1874 to Leiner is yet another patent showing a shaft-like body with looped ends, in this case apparently made by twisting two strands of wire, and in embodiments, adding a swab.

Finally, two design patents, U.S. Pat. No. Des. 444,556 to Estrem (Jul. 18, 2000), and U.S. Pat. No. Des. 428,489 to Huttner (Jul. 3, 2001) show looped ear curettes. Stamped plastic construction is not shown, and the length is excessive.

It will be appreciated that the circular loop shape provides better cleaning than the standard swab, however, wire, hard plastic or other stiffened ear cleaners offer the distinct possibility of damaging the ear drum.

These patents teach a variety of aspects of ear cleaners which bear improvement. First, they have hard metal or plastic loops which could damage an ear drum if pushed against it with sufficient force. Second, they show a length which allows such damage to occur. Third, they teach towards cylindrical or complex shapes of handles or overall tool, complex shapes which either require wire forming, plastic molding, and/or assembly, thus increasing costs.

### SUMMARY OF THE INVENTION

### GENERAL SUMMARY

The present invention teaches an improved ear cleaner consisting of one piece of solid flat plastic, inexpensively molded or stamped to an elongate oval shape with one aperture at each end to form a loop.

The present invention teaches that an improved ear cleaner may be made of a length such that it becomes difficult for the user to damage their ear drum. The invention further teaches that an improved ear cleaner may be made having a rounded shape and a soft consistency such that should the ear cleaning head (a thin and soft loop) impact the ear drum, it will bend rather than injure the ear drum.

The present invention further teaches a method of manufacturing the ear cleaner which provides a very inexpensive method of making a one piece ear cleaner requiring no further assembly after manufacture. The present invention further teaches that the ear cleaner may advantageously be stamped rather than molded for further decrease in cost of manufacture.

### SUMMARY IN REFERENCE TO CLAIMS

It is one aspect, advantage, embodiment and objective of the present invention to provide an ear cleaner comprising: a flat elongate body having at least one semicircular end; at least one aperture through the body, the aperture located at the at least one semicircular end and forming a first thin loop around the at least one aperture.

It is one aspect, advantage, embodiment and objective of the present invention to provide an ear cleaner wherein the flat elongate body further comprises: a second semicircular end; at least a second aperture through the body, the aperture located at the at second semicircular end, the second semicircular end forming a second thin loop around the second aperture.

It is one aspect, advantage, embodiment and objective of the present invention to provide an ear cleaner wherein the ear cleaner is made from a material selected from the list consisting of: polymers, fibers, layered sheets, foams, card boards, papers, plastics and combinations thereof.

It is one aspect, advantage, embodiment and objective of the present invention to provide an ear cleaner wherein the ear cleaner is made from a pliable material.

It is one aspect, advantage, embodiment and objective of the present invention to provide an ear cleaner wherein the semicircular end may have a planform selected from the group consisting of: circular, semicircular, elliptical, oval, portions of any of these shapes and combinations of these shapes.

It is one aspect, advantage, embodiment and objective of the present invention to provide an ear cleaner comprising: a flat elongate body having at least one semicircular end; at least one aperture through the body, the aperture located at the at least one semicircular end and forming a first thin loop around the at least one aperture.

It is one aspect, advantage, embodiment and objective of the present invention to provide an ear cleaner wherein the flat elongate body has a maximum length equal to the finger safety stop length of the target population.

It is one aspect, advantage, embodiment and objective of the present invention to provide an ear cleaner wherein the flat elongate body has a maximum length less than one centimeter plus the ear drum to ear exterior distance of the target population.

It is one aspect, advantage, embodiment and objective of the present invention to provide an ear cleaner wherein the flat elongate body further comprises: a second semicircular end; at least a second aperture through the body, the aperture located at the at second semicircular end, the second semicircular end forming a second thin loop around the second aperture.

It is one aspect, advantage, embodiment and objective of the present invention to provide an ear cleaner wherein the ear cleaner is made from a material selected from the list consisting of: polymers, fibers, layered sheets, foams, card boards, papers, plastics and combinations thereof.

It is one aspect, advantage, embodiment and objective of the present invention to provide an ear cleaner wherein the ear cleaner is made from a pliable material.

It is one aspect, advantage, embodiment and objective of the present invention to provide an ear cleaner wherein the ear cleaner is made entirely from the pliable material, with no part of the ear cleaner providing greater stiffness.

It is one aspect, advantage, embodiment and objective of the present invention to provide a method of making an ear cleaner comprising: die-cutting a sheet of material into at least one flat elongate body having at least one semicircular end and having at least one aperture through the body, the aperture located at the at least one semicircular end and forming a first thin loop around the at least one aperture.

It is one aspect, advantage, embodiment and objective of the present invention to provide a method of making an ear cleaner further comprising: freeing the ear cleaner from the sheet of material after the step of die cutting.

It is one aspect, advantage, embodiment and objective of the present invention to provide a method of making an ear cleaner, wherein the flat elongate body has a maximum length equal to the finger safety stop length of the target population.

It is one aspect, advantage, embodiment and objective of the present invention to provide a method of making an ear cleaner, wherein the flat elongate body has a maximum length less than one centimeter plus the ear drum to ear exterior distance of the target population.

It is one aspect, advantage, embodiment and objective of the present invention to provide a method of making an ear cleaner, wherein the flat elongate body further comprises: a second semicircular end; at least a second aperture through the body, the aperture located at the at second semicircular end, the second semicircular end forming a second thin loop around the second aperture.

It is one aspect, advantage, embodiment and objective of the present invention to provide a method of making an ear cleaner, wherein the ear cleaner is made from a material selected from the list consisting of: polymers, fibers, layered sheets, foams, card boards, papers, plastics and combinations thereof.

It is one aspect, advantage, embodiment and objective of the present invention to provide a method of making an ear cleaner, wherein the ear cleaner is made from a pliable material.

It is one aspect, advantage, embodiment and objective of the present invention to provide a method of making an ear cleaner, wherein the ear cleaner is made entirely from the pliable material, with no part of the ear cleaner providing greater stiffness.

### BRIEF DESCRIPTION OF THE DRAWINGS

FIG. 1 is a plan view of the invention.

FIG. 2 is a side view of the invention.

### DETAILED DESCRIPTION

The present invention is a flat, low cost ear cleaner which may be easily and cheaply constructed and which offers excellent safety features.

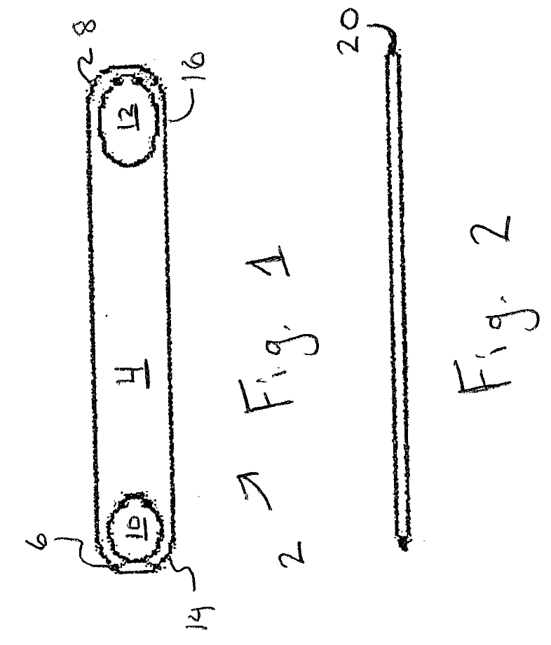

FIG. 1 is a plan view of the invention. Ear cleaner 2 has flat body 4. Rounded ends 6 and 8 have apertures 10 and 12 within them forming loops 14 and 16. Loops 14 and 16 may in planform be circular, semicircular, elliptical, or oval: all of these shapes, combination and portions of these shapes are considered to be “semicircular” as the term is used herein.

Body 4 has several important characteristics. Body 4 is flat, and may be made of an inexpensive, soft and pliable material suitable for the preferred methods of manufacture, and may be of a carefully selected length and width. Also, body 4 may have gently rounded edges.

The first characteristic, the flatness of body 4 is important to the preferred methods of manufacture discussed later. In addition, the flatness by itself, regardless of manufacture, provides a less expensive finished product. This flat body shape renders handling and use of the invention more easily accomplished than other shapes: a cylindrical shape, or a body made of a thin tube, twisted wires or thin section of Wood or plastic may be difficult to hold. On the other hand, a greatly contoured shape of body may engender confusion among users who are not yet familiar with the product and furthermore increases the cost of machine tool or mold preparation, increases the chances of rejected products during manufacture and thus greatly increases cost of the final product.

Secondly, body 4 may be made of inexpensive materials which permit the manufacture discussed below. Such materials should also be relatively soft and pliable. This is in contrast to known ear cleaners of metal, hard plastic, wooden, wire or paper construction stiffened by rolling or packing the paper tightly to form a rigid section of the ear cleaner, and which are all not pliable, as defined herein. The present invention teaches that a relatively soft material may provide good cleaning abilities, without altering the material in any way, at any portion of the length of the body or cleaning head (the loop) of the cleaning device. In particular, a softer material than wood or hard plastic greatly reduces the chances of accidental damage to the ear drum of the user, an increasing concern among doctors Soft plastic may be used, as may ordinary cardboard, cardboard surfaced with a paper product, layered materials of foam and soft plastic, or combinations of all of these. In use, when a user accidentally pushed to hard against any portion of the inner ear, such materials will “give”, flexing to prevent damage to the ear. In alternative embodiments, the material is sufficiently pliable to prevent damage to the ear drum regardless of how strongly the ear cleaner loop is pressed against the ear drum. For example, the loop may give under modest pressure, or under greater pressure, the loop may entirely collapse. Body 4 may flex along its length, as may loops 14 and 16, which being thinner will flex more quickly. Regarding manufacture, such relatively soft materials may be advantageously formed or purchased in sheets, thus providing the desirable flat shape of the final product.

Thirdly, the length and width of the finished product may be carefully selected. In general, the invention teaches an ear curette or ear cleaner having a length which does not allow the user to easily exert pressure on the ear drum. In use, the user may grasp the invention between thumb and index finger (or thumb and one or other fingers) and insert the end into the outer ear. However, the length of the invention may be carefully selected to prevent the user from being able to simultaneously grasp one end while pushing against the ear drum with the other end. The width of the invention may be selected to allow entry into the outer ear, but to prevent or impede entry further inwards as the channel of the outer ear narrows. The width may also be carefully selected to be large enough to provide a cleaning surface on the loop inside the ear which is relatively substantial in reference to the size of the ear.

Such safety measurements may be expressed in terms of the distance from the ear drum to the exterior of the ear. For example, in one presently preferred embodiment, the length of the ear cleaner may be selected to be that distance from ear drum to the exterior of the ear, called the ear channel depth, plus the additional length necessary for the user to hold the distal end between thumb and forefinger exactly at the entrance to the ear channel, that is a “two finger grip length”. The combination of the “two finger grip length” plus the ear channel depth is referred to in this application as the “finger safety stop length”, that is, the length at which the user's fingertips will touch the exterior of the ear and thus prevent the cleaning head of the invention from impacting the ear drum. In this preferred embodiment, the best mode now contemplated for carrying out the invention, there is no extra length available for the user to easily push the ear cleaning head (one of loop 14 or 16) against the ear drum. In alternative embodiments, the same length may be approximated as the distance from the ear drum to the exterior of the ear, plus approximately one centimeter.

Even more particularly, the length and width of the ear cleaner may be selected based upon a target population of users. For example, in certain geographical areas, the population may tend towards one average size, while in other geographical regions, the population may tend towards another size. Age is a very important factor the average size of the user and the user's ear, as are height, weight, ethnicity, gender and other factors. The length and width of the present invention may be adjusted based upon a target population. For example, differing sizes of the invention may be manufactured and in one geographic region, one size of ear cleaner may be sold while in another geographic region, a different size may be sold. As another example, a given region may be subdivided, for example on the basis of age, height, etc, with one or more sizes of ear cleaner being sold to each target demographic. This further promotes safety, as for example an ear cleaner optimized for an adult male population might be large enough to allow damage to the ears of a corresponding adult female population, and could well be large enough to be dangerous to the corresponding populations of children of various ages. In the presently preferred embodiment of the invention, a single “standard” target size is produced, however, other embodiments offer other sizes for clinical use, hospital use and so on. In other alternative embodiments, mass production of several standard target sizes may be possible.

Such differentiation of target user populations is aided by the extremely inexpensive methods of manufacture offered by this device.

Also, in the best mode now contemplated for carrying out the invention and preferred embodiment, the edges of body 4 may be gently rounded rather than angled. Thus, the edge of the ear cleaner does not present an inviting surface for the user to attempt to scrap across the interior of the ear. Also in the same embodiment the edges of loops 14 and 16 are gently rounded, specifically so that the user is not tempted to scrape right angled edges across the ear drum, thus increasing the chances of ear drum damage. In other embodiments in which the pliability and length of the ear cleaner of the present invention are found to present sufficient margins of safety, such angled edges may be employed the manufactured product. The edges may be two ninety degree angles, or the edge may be beveled, filleted or chamfered at any angles between zero and approximately one hundred and eighty degrees.

FIG. 2 is a side view of the preferred embodiment of the invention shown in FIG. 1, showing the rounded edges of the preferred embodiment. Body 4 has edge 20, which edge may be as discussed in the previous paragraph.

Method of Manufacture

The present device may advantageously be created by several methods of manufacture such as injection molding, machine processing, stamping, or advantageously, by die-cutting.

In the preferred embodiment and best mode now contemplated for carrying out of the invention, die-cutting or stamping of sheets of the soft-pliable material of the invention is used. The sheets of the desired raw material may be procured or manufactured in the desired length, width, thickness, and consistency. Such sheets, as previously discussed, may be several materials: soft plastic may be used, as may ordinary cardboard, cardboard surfaced with a paper product, layered materials of foam and soft plastic, or combinations of all of these. Such sheets are available at extremely low cost. The cost of the sheet may in embodiments be comparable to the cost of a single ear cleaner of complex three dimensional shape, yet advantageously and in the preferred embodiment of the invention, the size of the inexpensive sheets may be chosen to allow a large number of ear cleaners of the dimensions desired to be die-cut from a single sheet. The sheets may then be placed or fed into a die-cutting machine and the entire sheet cut into the large number of ear cleaners in a single operation. In a high speed machine, such die-cutting operations may convert great numbers of sheets into even greater numbers of ear cleaners in extremely short time. Such die cutting is obviously not available for production of ear cleaners having essentially cylindrical or complex body shapes. Yet the manufacturing advantages of die-cutting are quite impressive. The cost of tooling up for die cutting may be considerably lower than the cost creating molds or a custom built assembly line to machine ear cleaners of conventional types. This in turn allows the creation of greater numbers of sizes and shapes of the product, allowing greater market segmentation and thus increased user/consumer safety. Die-cutting may advantageously be employed with a range of polymer, fiber and layered sheets (plastics, papers, foams, card boards, and so on as mentioned earlier). It is also possible to die-cut sheets to as to automatically produce edges of the desired cross-section: gently rounded, squared, filleted, etc.

The ear cleaner may be freed from the sheet of material, or alternatively the ear cleaner may be left in the sheet of material. It is thus possible to manufacture and distribute the ear cleaner in sheets, which may contain a large number of ear cleaners per sheet. The end user need only break each ear cleaner free of the sheet of material in order to use it.

Method of Use

The present invention uses the superior loop shaped ear cleaning head, which allows the user to insert the invention into the ear and rotate it, causing the cleaning head to act like a “shovel”. Based upon clinical testing by Dr. Mendez, the cleaning action obtained is superior to that of a “swab” type cleaner.

Further in use, the user may insert the invention to the proper depth, as the invention is of the proper size to prevent over insertion, as discussed above. Further, during use of the invention, the invention may be pressed against the ear drum without fear of ear drum damage, as it has (in embodiments previously described) beveled or filleted edges, a degree of “give” in the cleaning head (the loop), and/or sufficient pliability to entirely prevent car damage, regardless of the force exerted by the user.

The disclosure is provided to allow practice of the invention by those skilled in the art without undue experimentation, including the best mode presently contemplated and the presently preferred embodiment. Nothing in this disclosure is to be taken to limit the scope of the invention, which is susceptible to numerous alterations, equivalents and substitutions without departing from the scope and spirit of the invention. The scope of the invention is to be understood from the appended claims.

What is claimed is:

1. An ear cleaner comprising:
a flat elongate body having at least one semicircular end;
at least one aperture through the body, the aperture located at the at least one semicircular end and forming a first thin loop around the at least one aperture.

2. The ear cleaner of claim 1, wherein the flat elongate body further comprises:
a second semicircular end;
at least a second aperture through the body, the aperture located at the at second semicircular end, the second semicircular end forming a second thin loop around the second aperture.

3. The car cleaner of claim 1, wherein the ear cleaner is made from a material selected from the list consisting of: polymers, fibers, layered sheets, foams, card boards, papers, plastics and combinations thereof.

4. The ear cleaner of claim 1, wherein the ear cleaner is made from a pliable material.

5. The ear cleaner of claim 1, wherein the semicircular end may have a planform selected from the group consisting of: circular, semicircular, elliptical, oval, portions of any of these shapes and combinations of these shapes.

6. An ear cleaner for a target population, the ear cleaner comprising:
a flat elongate body having at least one semicircular end;
at least one aperture through the body, the aperture located at the at least one semicircular end and forming a first thin loop around the at least one aperture.

7. The ear cleaner of claim 6, wherein the flat elongate body has a maximum length equal to the finger safety stop length of the target population.

8. The ear cleaner of claim 6, wherein the flat elongate body has a maximum length less than one centimeter plus the ear drum to ear exterior distance of the target population.

9. The ear cleaner of claim 8, wherein the flat elongate body further comprises:
a second semicircular end;
at least a second aperture through the body, the aperture located at the at second semicircular end, the second semicircular end forming a second thin loop around the second aperture.

10. The ear cleaner of claim 6, wherein the ear cleaner is made from a material selected from the list consisting of: polymers, fibers, layered sheets, foams, card boards, papers, plastics and combinations thereof.

11. The ear cleaner of claim 10, wherein the ear cleaner is made from a pliable material.

12. The ear cleaner of claim 11, wherein the ear cleaner is made entirely from the pliable material, with no part of the ear cleaner providing greater stiffness.

13. A method of making an ear cleaner comprising:
die-cutting a sheet of material into at least one flat elongate body having at least one semicircular end and having at least one aperture through the body, the aperture located at the at least one semicircular end and forming a first thin loop around the at least one aperture.

14. The method of making an ear cleaner of claim 13, further comprising:
freeing the ear cleaner from the sheet of material after the step of die cutting.

15. The method of making an ear cleaner of claim 13, wherein the flat elongate body has a maximum length equal to the finger safety stop length of the target population.

16. The method of making an ear cleaner of claim 13, wherein the flat elongate body has a maximum length less than one centimeter plus the ear drum to ear exterior distance of the target population.

17. The method of making an ear cleaner of claim 13, wherein the flat elongate body further comprises:
a second semicircular end;
at least a second aperture through the body, the aperture located at the at second semicircular end, the second semicircular end forming a second thin loop around the second aperture.

18. The method of making an ear cleaner of claim 13, wherein the ear cleaner is made from a material selected from the list consisting of: polymers, fibers, layered sheets, foams, card boards, papers, plastics and combinations thereof.

19. The method of making an ear cleaner of claim 13, wherein the ear cleaner is made from a pliable material.

20. The method of making an ear cleaner of claim 19, wherein the ear cleaner is made entirely from the pliable material, with no part of the ear cleaner providing greater stiffness.
